SEQUENCE LISTING (46 sequences)

SEQ ID NO: 1 - DNA, 20 bp, synthetic construct
  misc_feature 1..20: note = Primer
  source 1..20: mol_type = other DNA
cgtgcagaaatgcatcgact

SEQ ID NO: 2 - DNA, 20 bp, synthetic construct
  misc_feature 1..20: note = Primer
  source 1..20: mol_type = other DNA
atgtccacgatgtcgctctc

SEQ ID NO: 3 - DNA, 27 bp, synthetic construct
  misc_feature 1..27: note = Primer
  source 1..27: mol_type = other DNA
gacaaggtcactgtcaactactatgaa

SEQ ID NO: 4 - DNA, 22 bp, synthetic construct
  misc_feature 1..22: note = Primer
  source 1..22: mol_type = other DNA
ttgttcttctccacctctccat

SEQ ID NO: 5 - DNA, 20 bp, synthetic construct
  misc_feature 1..20: note = Primer
  source 1..20: mol_type = other DNA
cctaggccggcatgtctcta

SEQ ID NO: 6 - DNA, 20 bp, synthetic construct
  misc_feature 1..20: note = Primer
  source 1..20: mol_type = other DNA
ctcaggaaccgccccataaa

SEQ ID NO: 7 - DNA, 20 bp, synthetic construct
  misc_feature 1..20: note = Primer
  source 1..20: mol_type = other DNA
ggctacacaaccttcggcat

SEQ ID NO: 8 - DNA, 20 bp, synthetic construct
  misc_feature 1..20: note = Primer
  source 1..20: mol_type = other DNA
gctgctttcacctgtagggt

SEQ ID NO: 9 - DNA, 20 bp, synthetic construct
  misc_feature 1..20: note = Primer
  source 1..20: mol_type = other DNA
gtggctagcttggtaagccc

SEQ ID NO: 10 - DNA, 21 bp, synthetic construct
  misc_feature 1..21: note = Primer
  source 1..21: mol_type = other DNA
tgcacacttgctgatgtccaa

SEQ ID NO: 11 - DNA, 20 bp, synthetic construct
  misc_feature 1..20: note = Primer
  source 1..20: mol_type = other DNA
tccctgaagatgcctaccca

SEQ ID NO: 12 - DNA, 21 bp, synthetic construct
  misc_feature 1..21: note = Primer
  source 1..21: mol_type = other DNA
cacccttgggcacatcactgt

SEQ ID NO: 13 - DNA, 18 bp, synthetic construct
  misc_feature 1..18: note = Primer
  source 1..18: mol_type = other DNA
gcaggctgctggagaagg

SEQ ID NO: 14 - DNA, 20 bp, synthetic construct
  misc_feature 1..20: note = Primer
  source 1..20: mol_type = other DNA
cgctgtagacatcggtccag

SEQ ID NO: 15 - DNA, 20 bp, synthetic construct
  misc_feature 1..20: note = Primer
  source 1..20: mol_type = other DNA
agtccaacaccagcatgtcg

SEQ ID NO: 16 - DNA, 21 bp, synthetic construct
  misc_feature 1..21: note = Primer
  source 1..21: mol_type = other DNA
cctcaggcactgacccataaa

SEQ ID NO: 17 - DNA, 22 bp, synthetic construct
  misc_feature 1..22: note = Primer
  source 1..22: mol_type = other DNA
gtttagggtcagacagtcctgg

SEQ ID NO: 18 - DNA, 20 bp, synthetic construct
  misc_feature 1..20: note = Primer
  source 1..20: mol_type = other DNA
ggctacacaaccttcggcat

SEQ ID NO: 19 - DNA, 20 bp, synthetic construct
  misc_feature 1..20: note = Primer
  source 1..20: mol_type = other DNA
cctgacctgttgcggatgat

SEQ ID NO: 20 - DNA, 20 bp, synthetic construct
  misc_feature 1..20: note = Primer
  source 1..20: mol_type = other DNA
agcaggtccccttgatttgg

SEQ ID NO: 21 - DNA, 22 bp, synthetic construct
  misc_feature 1..22: note = Primer
  source 1..22: mol_type = other DNA
agatccacagcgatatccagac

SEQ ID NO: 22 - DNA, 22 bp, synthetic construct
  misc_feature 1..22: note = Primer
  source 1..22: mol_type = other DNA
accgaagagacaacggcacact

SEQ ID NO: 23 - DNA, 22 bp, synthetic construct
  misc_feature 1..22: note = Primer
  source 1..22: mol_type = other DNA
cgaagactacagttctgccatt

SEQ ID NO: 24 - DNA, 22 bp, synthetic construct
  misc_feature 1..22: note = Primer
  source 1..22: mol_type = other DNA
gacgtttcagaggttctcagag

SEQ ID NO: 25 - DNA, 22 bp, synthetic construct
  misc_feature 1..22: note = Primer
  source 1..22: mol_type = other DNA
gcaactgttcctgaactcaact

SEQ ID NO: 26 - DNA, 21 bp, synthetic construct
  misc_feature 1..21: note = Primer
  source 1..21: mol_type = other DNA
atcttttggggtccgtcaact

SEQ ID NO: 27 - DNA, 22 bp, synthetic construct
  misc_feature 1..22: note = Primer
  source 1..22: mol_type = other DNA
cttccatccagttgccttcttg

SEQ ID NO: 28 - DNA, 23 bp, synthetic construct
  misc_feature 1..23: note = Primer
  source 1..23: mol_type = other DNA
aattaagcctccgacttgtgaag

SEQ ID NO: 29 - DNA, 20 bp, synthetic construct
  misc_feature 1..20: note = Primer
  source 1..20: mol_type = other DNA
gtgaaccccagaccagactg

SEQ ID NO: 30 - DNA, 22 bp, synthetic construct
  misc_feature 1..22: note = Primer
  source 1..22: mol_type = other DNA
cctggaacacgtttctgaaaga

SEQ ID NO: 31 - DNA, 20 bp, synthetic construct
  misc_feature 1..20: note = Primer
  source 1..20: mol_type = other DNA
tgctgaccccaagaaggaat

SEQ ID NO: 32 - DNA, 21 bp, synthetic construct
  misc_feature 1..21: note = Primer
  source 1..21: mol_type = other DNA
gaagtgcttgaggtggttgtg

SEQ ID NO: 33 - DNA, 21 bp, synthetic construct
  misc_feature 1..21: note = Primer
  source 1..21: mol_type = other DNA
agatctctgcagctgccctca

SEQ ID NO: 34 - DNA, 22 bp, synthetic construct
  misc_feature 1..22: note = Primer
  source 1..22: mol_type = other DNA
ggagcacttgctgctggtgtag

SEQ ID NO: 35 - DNA, 21 bp, synthetic construct
  misc_feature 1..21: note = Primer
  source 1..21: mol_type = other DNA
ctttcagcatccaagtgtggg

SEQ ID NO: 36 - DNA, 20 bp, synthetic construct
  misc_feature 1..20: note = Primer
  source 1..20: mol_type = other DNA
atgctatagcctcctcgacc

SEQ ID NO: 37 - DNA, 21 bp, synthetic construct
  misc_feature 1..21: note = Primer
  source 1..21: mol_type = other DNA
ccgtcggaggagactacaaag

SEQ ID NO: 38 - DNA, 20 bp, synthetic construct
  misc_feature 1..20: note = Primer
  source 1..20: mol_type = other DNA
gcagaggtccgtggattctt

SEQ ID NO: 39 - DNA, 20 bp, synthetic construct
  misc_feature 1..20: note = Primer
  source 1..20: mol_type = other DNA
gggcgtaaacgcttcgagat

SEQ ID NO: 40 - DNA, 20 bp, synthetic construct
  misc_feature 1..20: note = Primer
  source 1..20: mol_type = other DNA
tcaggtagctggagtgagcc

SEQ ID NO: 41 - DNA, 20 bp, synthetic construct
  misc_feature 1..20: note = Primer
  source 1..20: mol_type = other DNA
gagtctccatgcagctacgg

SEQ ID NO: 42 - DNA, 20 bp, synthetic construct
  misc_feature 1..20: note = Primer
  source 1..20: mol_type = other DNA
ttctcttcaatccggtggcg

SEQ ID NO: 43 - DNA, 20 bp, synthetic construct
  misc_feature 1..20: note = Primer
  source 1..20: mol_type = other DNA
ggcctcaccccatttgatgt

SEQ ID NO: 44 - DNA, 23 bp, synthetic construct
  misc_feature 1..23: note = Primer
  source 1..23: mol_type = other DNA
catgttccagtatgactccactc

SEQ ID NO: 45 - DNA, 24 bp, synthetic construct
  misc_feature 1..24: note = Primer
  source 1..24: mol_type = other DNA
ttcgacagtcagccgcatcttctt

SEQ ID NO: 46 - DNA, 24 bp, synthetic construct
  misc_feature 1..24: note = Primer
  source 1..24: mol_type = other DNA
gcccaatacgaccaaatccgttga

(19) United States
(12) Patent Application Publication
(10) Pub. No.: US 2023/0203174 A1
(43) Pub. Date: Jun. 29, 2023

(54) COMPOSITION FOR PREVENTING OR ALLEVIATING CELLULAR SENESCENCE

(71) Applicant: SEOUL NATIONAL UNIVERSITY R&DB FOUNDATION, Seoul (KR)

(72) Inventors: Hyun Mo RYOO, Seoul (KR); Hyun Jung KIM, Seoul (KR); Woo Jin KIM, Seoul (KR)

(73) Assignee: SEOUL NATIONAL UNIVERSITY R&DB FOUNDATION, Seoul (KR)

(21) Appl. No.: 18/057,519

(22) Filed: Nov. 21, 2022

(30) Foreign Application Priority Data
Dec. 13, 2021 (KR) 10-2021-0177439

(51) Int. Cl. C07K 16/28; G01N 33/68; G01N 33/564; C12N 9/78

(52) U.S. Cl. CPC C07K 16/2866; G01N 33/6854; G01N 33/564; C12N 9/78; G01N 2333/978; G01N 2800/102; C07K 2317/76; A61K 2039/505

(57) ABSTRACT

Uses and a composition including a peptidylarginine deiminase 2 (PADI2) as an active ingredient are disclosed. A peptidylarginine deiminase 2 (PADI2) is useful for preventing or alleviating cellular senescence.

### CROSS REFERENCE TO RELATED APPLICATION

This application claims priority from Korean Patent Application No. 10-2021-0177439 filed Dec. 13, 2021 in the Korea Intellectual Property Office, of which the entire content is incorporated herein by reference.

### INCORPORATION BY REFERENCE OF SEQUENCE LISTING

The content of the electronically submitted sequence listing, file name: Sequence_Listing_As_Filed.xml; size: 58,409 bytes; and date of creation: Nov. 18, 2022, filed herewith, is incorporated herein by reference in its entirety.

### BACKGROUND

### 1. Field of the Invention

The present invention relates to a composition for preventing or alleviating cellular senescence, and more particularly, to a composition for preventing or alleviating osteoblast senescence during aging.

### 2. Discussion of Related Art

Biological aging can be defined as a gradual deterioration of body functions due to the accumulation of damages in cells and tissues. If reactive oxygen species (ROS), which are inevitably generated in the normal metabolic process of cells, are not removed properly and accumulate inside and outside cells, it can cause damages to intracellular molecules. The accumulation of these damaged molecules can be a major cause of senescence and age-related diseases.

ROS generated exogenously or endogenously during normal metabolic processes are known to play an important role in a senescence process (Sohal R S et al. 1996). Although ROS is normally maintained at a balanced level by a cellular antioxidant system, a oxidative stress caused by an imbalance of ROS can promote cellular senescence, and as senescence progresses, the accumulated senescent cells affect a normal physiological function of tissues, causing gradual functional degeneration. Abnormal ROS in bone tissue due to aging negatively affects the maintenance of bone homeostasis (Wauquier F et al. 2009; Altindag O et al. 2008).

That is, it inhibits osteoblast activity and activates osteoclast activity to induce osteopenia. Studies on the mechanism of bone loss caused by excessive ROS have been mainly focused on the mechanism of activating osteoclasts, and many studies have not been conducted on the effect on differentiation and function of osteoblasts.

Peptidylarginine deiminases (PADIs) are enzymes that convert positively charged peptidyl-arginine residues into neutral peptidyl-citrulline through a hydrolysis process called citrullination or deamination (Vossenaar E R et al. 2003; Bicker K L & Thompson P R 2013).

These post-translational modifications can have a significant impact on a structure and a function of a target protein (Vossenaar E R et al. 2003). In mammals, five PADI enzymes consisting of PADI 1, 2, 3, 4 and 6 are known to exist, and their expression patterns and functions are known to be tissue-specific (Vossenaar E R et al. 2003; Wang S & Wang Y, 2013). Citrullation has been reported to play an important role in cellular processes such as inflammatory immune responses and regulation of gene expression (Sanchez-Pernaute O et al. 2013; Zhang X et al. 2012). Recently, it was reported that PADI2-induced citrullation is required for oligodendrocyte differentiation and myelination (Falcao A M et al. 2019). However, roles and functions of PADI isoforms including PADI2 in senescence have not been reported so far.

Senescent cells secrete inflammatory cytokines, growth factors, chemokines, and proteases, which are referred to as the age-associated secretory phenotypes (SASPs) (Perez-Mancera P A et al. 2014). These SASP factors show various compositions according to cell types or senescence-inducing factors. Secretion of SASP by senescent cells affects the tissue microenvironment in an autocrine and paracrine manner, which allows senescence to propagate to surrounding normal cells (Acosta J C et al. 2013). CCL-2 (MCP-1), CCL-5 (RANTES), CCL-7 (MCP-3), CXCL-1 and CXCL12 (SDF-1), including proinflammatory cytokines including Interleukin-1a (IL-1a), Interleukin-6 (IL-6), and interleukin-8 (IL-8), have also been identified as important SASP components secreted by senescent cells (Coppe J P et al. 2010). It has been reported that NFκB p65/RelA is activated during senescence or DNA repair response as a master regulator regulating an expression of factors constituting SASP to regulate their expression (Chien Y et al. 2011).

### SUMMARY OF THE INVENTION

The present invention is directed to utilizing a decrease of PADI2, increases in CCL2, CCL5 and CCL7, and NFκB activation as biomarkers for senescence and age-related diseases, and as surrogate markers capable of evaluating efficacies of anti-senescence agents and therapeutic agents for age-related diseases.

The present invention is directed to utilizing an antibody or siRNA capable of inhibiting CCL2, CCL5, and CCL7 for treatment of age-related diseases caused by decrease of PADI2.

The present invention is directed to utilizing inhibitors of NFκB signaling pathway for treatment of senescence and age-related diseases caused by decrease of PADI2.

Specifically, as a composition for preventing or alleviating cellular senescence according to one embodiment of the present invention, the composition including the PADI2 prevents or alleviates cellular senescence by inhibiting secretion of inflammatory cytokines, growth factors, chemokines, or proteases in cells or inhibiting an activity of NFκB.

The present invention is directed to promoting a differentiation function of osteoblasts by providing PADI2 to osteoblasts.

The present invention is directed to alleviating or treating senescence or age-related diseases caused by a decrease of PADI2 through antibodies of CCL2, CCL5 and CCL7.

The present invention is directed to alleviating or treating senescence or age-related diseases caused by a decrease of PADI2 through an NFκB inhibitor.

The present invention is directed to alleviating or treating senescence or age-related diseases caused by a decrease of PADI2 by administering antibodies of CCL2, CCL5 and CCL7 to a subject.

The present invention is directed to alleviating or treating senescence or age-related diseases caused by a decrease of PADI2 by administering an NFκB inhibitor to a subject.

The present invention is directed to providing a diagnostic kit capable of diagnosing a decrease of PADI2 using antibodies of CCL2, CCL5 and CCL7.

The present invention is directed to providing a diagnostic kit capable of diagnosing a decrease of PADI2 using an NFκB inhibitor.

The objects of the present invention are not limited to those mentioned above, and further objects which are not mentioned will be apparent to those skilled in the art from the following description.

According to one aspect of the present invention, as a composition for preventing or alleviating cellular senescence, a composition including PADI2 may be provided.

Here, the composition may inhibit secretion of inflammatory cytokines, growth factors, chemokines, or proteases in cells.

Here, the composition may inhibit an activity of NFκB.

According to another aspect of the present invention, as a composition for improving a differentiation function of osteoblasts, a composition including PADI2 may be provided.

According to another aspect of the present invention, as a composition for alleviating or treating senescence or age-related diseases caused by a decrease of PADI2, a composition including antibodies of CCL2, CCL5 and CCL7 may be provided.

According to another aspect of the present invention, as a composition for alleviating or treating senescence or age-related diseases caused by a decrease of PADI2, a composition including an NFκB inhibitor may be provided.

According to another aspect of the present invention, there may be provided a method for alleviating or treating senescence or age-related diseases caused by a decrease of PADI2, comprising administering antibodies of CCL2, CCL5 and CCL7 to a non-human subject.

According to another aspect of the present invention, there may be provided a method for alleviating or treating senescence or age-related diseases caused by a decrease of PADI2, comprising administering an NFκB inhibitor to a non-human subject.

According to another aspect of the present invention, a kit including detectable labeled antibodies of CCL2, CCL5 and CCL7 as a diagnostic kit for a decrease of PADI2 may be provided.

According to another aspect of the present invention, a kit including a detectable labeled NFκB inhibitor as a diagnostic kit for a decrease of PADI2 may be provided.

### BRIEF DESCRIPTION OF THE DRAWINGS

FIGS. 1A-1D show experimental results confirming that excessive ROS promotes the senescence of osteoblasts and inhibits osteoblast differentiation.

FIGS. 2A-2D are results of verifying whether excessive ROS reduces PADI2 in both mouse-derived and human-derived mesenchymal cells.

FIGS. 3A-3D show whether cellular senescence is promoted when Padi2 is knocked down by transfection of Padi2 siRNA into MC3T3-E1 cells to confirm whether the decrease of PADI2 caused by excessive ROS directly induces promotion of cellular senescence.

FIGS. 4A-4E show that cellular senescence is promoted, and a differentiation function is inhibited in PADI2 knockout cells by CRISPR-Cas9 gene editing technology compared to wild type control cells.

FIG. 5 shows that factors secreted from PADI2-knockdown cells promote cellular senescence.

FIGS. 6A-6E show that PADI2 knockdown promotes the expression and secretion of SASP factors (CCL2, CCL5, and CCL7).

FIGS. 7, 8A, and 8B show the restoration of cell senescence and functional degradation when CCL2, CCL5, and CCL7 are inhibited using an antibody or siRNA.

FIGS. 9A-9D and 10A-10C show that excessive ROS and PADI2 knockdown activate NFκB, thereby increasing CCL2, CCL5, and CCL5 expressions.

FIGS. 11A-11C show the restoration of cellular senescence and functional degradation of osteoblasts due to excessive ROS and PADI2 knockdown upon the inhibition of NFκB signaling.

FIG. 12 is a comparison result of PADI2 mRNA expression in mesenchymal stem cells (hMSCs) derived from young and elderly humans.

FIG. 13 is a comparison result of PADI2 protein level in young and old mouse cranial bone tissues.

FIG. 14 is a schematic diagram describing the mechanism of cellular senescence and functional deterioration of osteoblasts by the decrease of PADI2.

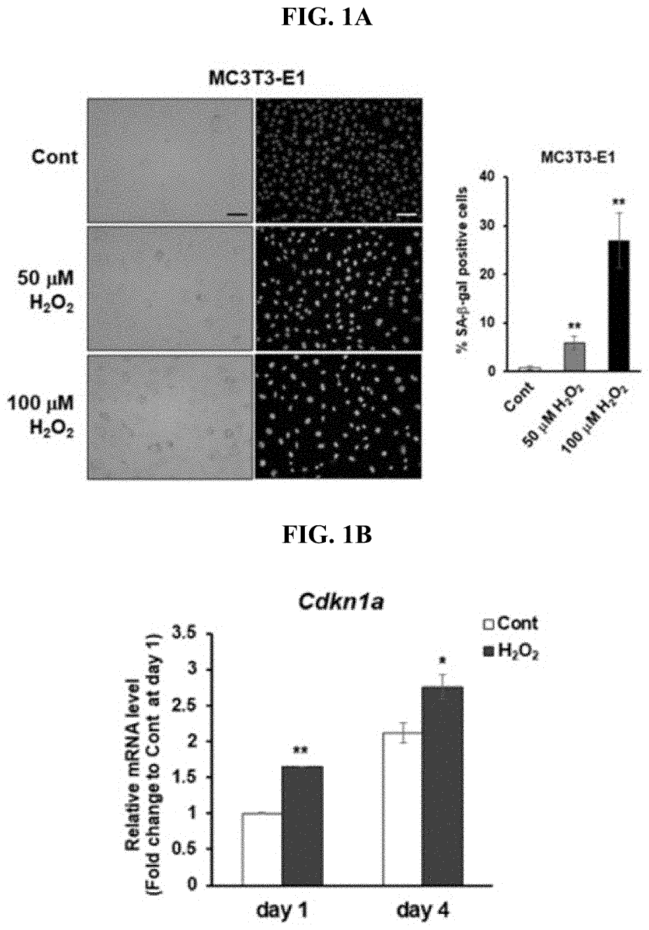

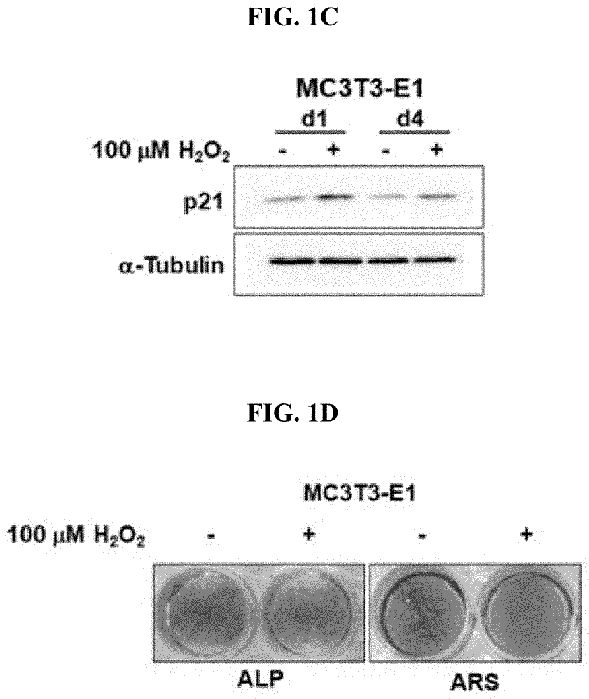

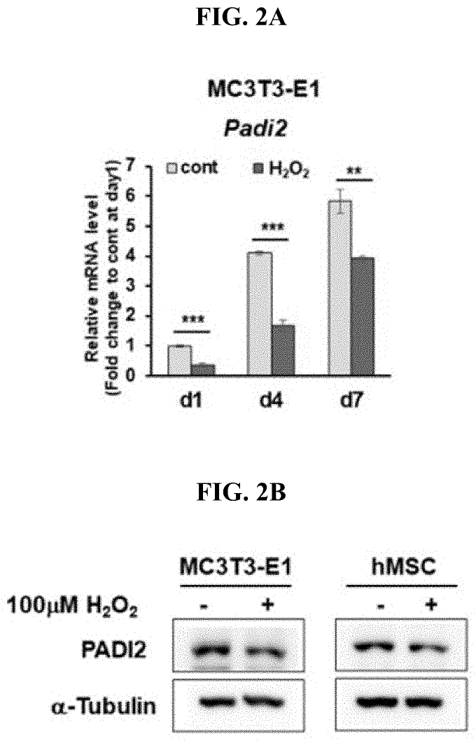

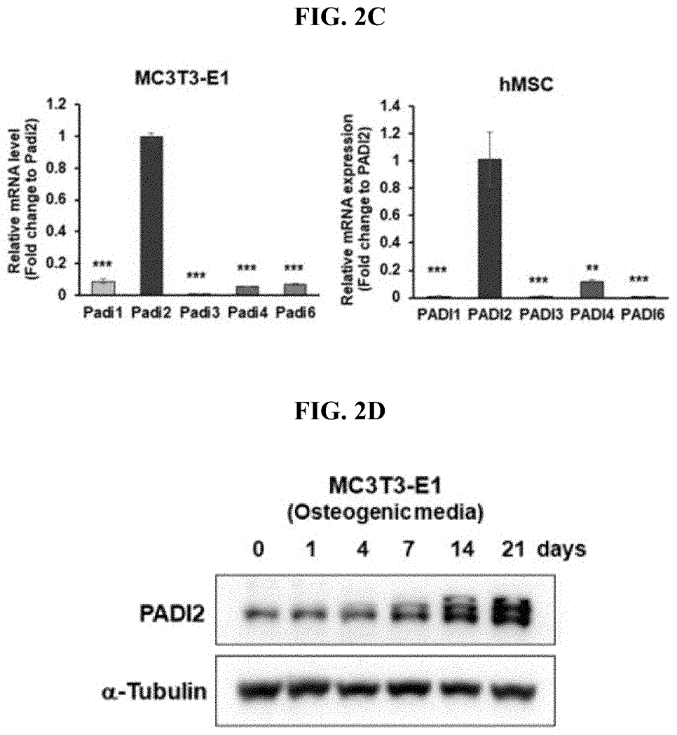

### DETAILED DESCRIPTION OF EXEMPLARY EMBODIMENTS

Objects and advantages of the present invention, and technical configurations for achieving them, will become apparent with reference to the embodiments described in detail below in conjunction with the accompanying drawings. In the description of the present invention, when it is determined that a specific description of a known function or configuration may unnecessarily obscure the gist of the present invention, the detailed description thereof will be omitted. And the following terms are defined terms in consideration of the donation in the present invention, which may vary according to the user, the intent or practice of the operator, etc.

However, the present invention is not limited to the embodiments disclosed below, but may be embodied in various different forms. The examples are merely provided to complete the disclosure of the present invention and to fully illuminate the scope of the invention to those skilled in the art, and the invention is only defined by the scope of the claims. The definition should therefore be made on the basis of the disclosure throughout this specification.

In the description of the present invention, “individual” means a mammal, preferably a human. In one embodiment, the individual may be “patient”, that is a warm-blooded animal, more preferably a human who is awaiting or receiving medical treatment, or is, will be, or is being followed for, a subject of the medical treatment for progression or cure of cellular senescence or cellular damage due to decrease of PADI2.

In one embodiment, the individual is an adult (for example, 18 years of age or older). In other embodiments, the individual is a child (for example, an individual 18 years of age or younger). In one embodiment, the individual is male. In other embodiments, the subject is female.

In the description of the present invention, “treatment” or “alleviation” means a therapeutic action performed and not performed for the purpose of improving a patient's condition, for example, the action aims at slowing (relieving) symptoms or preventing or alleviating progress thereof of due to functional degradation of osteoblasts or body-constituting various cells. When a patient administered the composition according to the present invention is as described below, a disease resulting from cellular senescence in an individual or mammal is successfully “treated”: amelioration to some extent of one or more symptoms associated with cellular senescence; reduced morbidity; reduced mortality; or an observable and/or measurable decrease or disappearance of one or more of the improvements in quality of life. The parameters for evaluating the successful treatment and amelioration of the injury can be readily measured by routine methods familiar to the physician.

Meanwhile, when referring to measurable values such as amount, dosage, time, temperature and the like of the composition, “about” and “approximately” are intended to include variations of 20%, 10%, 5%, 1%, 0.5%, or 0.1% of a specified value.

In the description of the present invention, an oxygen steady state (normoxia) refers to tissue or physiological oxygen levels. In one embodiment of the present invention, culturing the cells at the oxygen steady state, or at physiological oxygen levels means culturing at an oxygen level of about 3% to about 6%, preferably at an oxygen level of about 5%.

Hereinafter, examples of the present invention will be described in detail.

### EXAMPLES

Reagent and Antibody

30% hydrogen peroxide (H1009), X-Gal (B4252), potassium hexacyanoferrate (III) (208019), potassium ferrocyanide (P9387) were purchased from Sigma-Aldrich (St. Louis, Mo., USA). Bay11-7082 (S2913) was purchased from Selleck Chemicals (Houston, Tex., USA). Antibodies used in the present invention are as follows: Padi2 (66386-1-Ig; Proteintech, Rosemont, Ill., USA), α-tubulin (sc-8035; Santa Cruz Biotechnology, Inc., Dallas, Tex., USA), β-actin (sc-47778; Santa Cruz Biotechnology.), LaminA/C (sc-376248; Santa Cruz Biotechnology), p21 (#2947; Cell Signaling Technology, Inc., Danvers, Mass., USA), a mouse CCL2 antibody (AB-479-NA; R&D Systems, Inc, Minneapolis, Minn., USA), a mouse CCL5 antibody (AF478; R&D Systems), a mouse CCL7 antibody (AF-456-NA; R&D Systems) and a normal goat IgG control (AB-108-C, R&D Systems, Inc). The siRNA used in the present invention was purchased from Origene (R&D Systems, Inc): Padi2 siRNA (SR418983B and C), RelA siRNA (SR427138A), Ccl2 siRNA (SR403037C), Ccl5 siRNA (SR400775A), Ccl7 siRNA (SR400957C), and negative control siRNA (SR30004).

Cell Culture

MC3T3-E1 cells were cultured in an α-MEM medium supplemented with 10% fetal bovine serum (FBS) containing 100 U/mL penicillin and 100 μg/mL streptomycin. To induce osteoblast differentiation, an α-MEM differentiation medium supplemented with 10 mM β-glycerophosphate and 50 μg/mL ascorbic acid was used. Human mesenchymal stem cells were purchased from STEMCELL Technologies (Vancouver, Canada) and cultured according to a manufacturer's protocol.

Senescence Associated-β-Galactosidase (SA-β-gal) Staining

SA-β-gal staining was performed with reference to the paper (2009) published by the Debacq-Chainiaux group. Cells cultured on coverslips were fixed in 4% paraformaldehyde at room temperature for 6 min. After washing cells three times with PBS, the cells were reacted for 16-24 hours in a staining solution [40 mM Na₂HPO₄, pH 6.0, 150 mM NaCl, 2 mM MgCl₂, 5 mM K3Fe(CN)6, 5 mM K₄Fe(CN)₆ and 1 mg/ml X-Gal]. After washing 3 times with PBS, it was permeabilized with PBS containing 0.2% Triton X-100 at room temperature for 15 minutes. For nuclear staining, a mounting solution containing 4′,6-diamidino-2-phenylindole (DAPI) was used (AR-6501-01; ImmunoBioScience Corp., Mukilteo, Wash., USA). Images were acquired using an Eclipse TS-100 inverted microscope (Nikon). Using the ImageJ program, nucleus (DAPI) and SA-b-gal positive cells were counted in each image and compared as an average of % SA-β-gal positive cells (SA-β-gal positive cells/nuclear number×100) using the ImageJ program using 10 images of different parts per group.

### REFERENCES

Debacq-Chainiaux F, Erusalimsky J D, Campisi J, Toussaint O (2009) Protocols to detect senescence-associated beta-galactosidase (SA-beta-gal) activity, a biomarker of senescent cells in culture and in vivo. Nat Protoc 4 (12):1798-1806.

Western Blot Analysis

Protein was extracted using a PRO-PREP protein extraction solution (Cat #17081, iNtRON, Korea) according to a manufacturer's protocol. The same amount of protein was transferred to a polyvinylidene fluoride (PVDF) membrane after sodium dodecyl sulfate polyacrylamide gel electrophoresis (SDS-PAGE). After blocking with 5% non-fat milk, membranes were blotted in primary antibody solution at 4° C. in a refrigerator overnight. A membrane reacted with a primary antibody was washed 3 times every 5 minutes with PBS-0.05% Tween 20 solution, then reacted with a secondary antibody for 1-2 hours at room temperature, and then the membrane was washed 3 times with PBS-0.05% Tween 20 solution for 5 minutes. Thereafter, the images were acquired on an ImageQuant LAS4000 Mini (GE Healthcare) using chemiluminescence (Clarity™ Western ECL Substrate, #170-5060; Bio-Rad). α-Tubulin or β-actin was used as a protein loading control.

RNA Preparation and Quantitative Real-Time PCR (RT-qPCR)

Total RNA was isolated using the RNeasy Mini Kit (Qiagen, Hilden, Germany). Total RNA (1 μg) was reverse transcribed into cDNA using PrimeScript RT Master Mix (Perfect Real Time) (RR036A; TaKaRa, Japan) and a real-time PCR was performed on an STEPONEPLUS™ Real-Time PCR System (APPLIED BIOSYSTEMS™) instr μMent using TB GREEN® PREMIX EX TAQ™ (RR420A; TaKaRa). After denaturation at 95° C. for 1 min, the real-time PCR was performed in 40 cycles at 95° C. for 15 sec and 60° C. for 30 sec. Primers used in the present invention are listed in Table 1.

TABLE 1
Primer sequence (forward, FOR; reverse, REV)
used for RT-qPCR

| Gene | Sequences for primers | SEQ ID NO |
|---|---|---|
| Padi1 | FOR: CGTGCAGAAATGCATCGACT | SEQ ID NO: 1 |
| | REV: ATGTCCACGATGTCGCTCTC | SEQ ID NO: 2 |
| Padi2 | FOR: GACAAGGTCACTGTCAACTACT | SEQ ID NO: 3 |
| | ATGAA | |
| | REV: TTGTTCTTCTCCACCTCTCCAT | SEQ ID NO: 4 |
| Padi3 | FOR: CCTAGGCCGGCATGTCTCTA | SEQ ID NO: 5 |
| | REV: CTCAGGAACCGCCCCATAAA | SEQ ID NO: 6 |
| Padi4 | FOR: GGCTACACAACCTTCGGCAT | SEQ ID NO: 7 |
| | REV: GCTGCTTTCACCTGTAGGGT | SEQ ID NO: 8 |
| Padi6 | FOR: GTGGCTAGCTTGGTAAGCCC | SEQ ID NO: 9 |
| | REV: TGCACACTTGCTGATGTCCAA | SEQ ID NO: 10 |
| PADI1 | FOR: TCCCTGAAGATGCCTACCCA | SEQ ID NO: 11 |
| | REV: CACCCTTGGGCACATCACTGT | SEQ ID NO: 12 |
| PADI2 | FOR: GCAGGCTGCTGGAGAAGG | SEQ ID NO: 13 |
| | REV: CGCTGTAGACATCGGTCCAG | SEQ ID NO: 14 |
| PADI3 | FOR: AGTCCAACACCAGCATGTCG | SEQ ID NO: 15 |
| | REV: CCTCAGGCACTGACCCATAAA | SEQ ID NO: 16 |
| PADI4 | FOR: GTTTAGGGTCAGACAGTCCTGG | SEQ ID NO: 17 |
| | REV: AGATGTGAGTAGTGGCACATGC | SEQ ID NO: 18 |
| PADI6 | FOR: CCTGACCTGTTGCGGATGAT | SEQ ID NO: 19 |
| | REV: AGCAGGTCCCCTTGATTTGG | SEQ ID NO: 20 |
| Cdkn1α | FOR: AGATCCACAGCGATATCCAGAC | SEQ ID NO: 21 |
| | REV: ACCGAAGAGACAACGGCACACT | SEQ ID NO: 22 |
| Il1α | FOR: CGAAGACTACAGTTCTGCCATT | SEQ ID NO: 23 |
| | REV: GACGTTTCAGAGGTTCTCAGAG | SEQ ID NO: 24 |
| Il1β | FOR: GCAACTGTTCCTGAACTCAACT | SEQ ID NO: 25 |
| | REV: ATCTTTTGGGGTCCGTCAACT | SEQ ID NO: 26 |
| Il6 | FOR: CTTCCATCCAGTTGCCTTCTTG | SEQ ID NO: 27 |
| | REV: AATTAAGCCTCCGACTTGTGAA | SEQ ID NO: 28 |
| | G | |
| Il18 | FOR: GTGAACCCCAGACCAGACTG | SEQ ID NO: 29 |
| | REV: CCTGGAACACGTTTCTGAAAGA | SEQ ID NO: 30 |
| Ccl2 | FOR: TGCTGACCCCAAGAAGGAAT | SEQ ID NO: 31 |
| | REV: GAAGTGCTTGAGGTGGTTGTG | SEQ ID NO: 32 |
| Ccl5 | FOR: AGATCTCTGCAGCTGCCCTCA | SEQ ID NO: 33 |
| | REV: GGAGCACTTGCTGCTGGTGTAG | SEQ ID NO: 34 |
| Ccl7 | FOR: CTTTCAGCATCCAAGTGTGGG | SEQ ID NO: 35 |
| | REV: ATGCTATAGCCTCCTCGACC | SEQ ID NO: 36 |
| Igfbp6 | FOR: CCGTCGGAGGAGACTACAAAG | SEQ ID NO: 37 |
| | REV: GCAGAGGTCCGTGGATTCTT | SEQ ID NO: 38 |
| Trp53 | FOR: GGGCGTAAACGCTTCGAGAT | SEQ ID NO: 39 |
| | REV: TCAGGTAGCTGGAGTGAGCC | SEQ ID NO: 40 |
| RelA | FOR: GAGTCTCCATGCAGCTACGG | SEQ ID NO: 41 |
| | REV: TTCTCTTCAATCCGGTGGCG | SEQ ID NO: 42 |
| Gapdh | FOR: GGCCTCACCCCATTTGATGT | SEQ ID NO: 43 |
| | REV: CATGTTCCAGTATGACTCCACT | SEQ ID NO: 44 |
| | C | |
| GAPDH | FOR: TTCGACAGTCAGCCGCATCTTC | SEQ ID NO: 45 |
| | TT | |
| | REV: GCCCAATACGACCAAATCCGTT | SEQ ID NO: 46 |
| | GA | |

Enzyme-Linked Immunosorbent Assay (ELISA)

The levels of CCL2, CCL5, and CCL7 secreted into a medium during cell culture were measured by ELISA. For this purpose, a mouse CCL2 ELISA kit (ab208979; Abcam, Cambridge, UK), a mouse CCL5 ELISA kit (ab100739; Abcam) and a mouse CCL7 ELISA kit (ab205571; Abcam) were purchased and performed according to a manufacturer's protocol.

Nuclear-Cytoplasmic Fractionation

After collecting cells with PBS, a cell pellet was equally divided into two tubes for whole-cell lysate and cell fractionation. The whole-cell lysate prepared with 100 μL of lysis buffer composed of 25 mM Tris-HCl pH 7.4, 150 mM NaCl, 1% NP-40, 1 mM EDTA, 5% glycerol, protease inhibitor cocktail (Roche, #11-836-153-001), phosphatase inhibitor cocktail 2 and 3 (Sigma-Aldrich, P5726 and P004 SEQ ID NO: 4). The nuclear-cytoplasmic fraction was isolated using NEPER™ nuclear and cytoplasmic extraction reagents (78855; Life Technologies, Carlsbad, Calif., USA) according to a manufacturer's protocol. Protein concentration was determined using the Pierce™ BCA Protein Assay Kit (Thermo Fisher Scientific). The whole-cell lysate, a cytoplasmic extract and a nuclear extract were separated by SDS-PAGE, transferred to PVDF membrane, and then Western blot analysis was performed with designated primary and secondary antibodies. Tubulin and Lamin A/C were used as cytoplasmic and nuclear markers, respectively.

Immunofluorescence and Confocal Microscope

Cells cultured on coverslips were fixed in 4% paraformaldehyde, blocked with PBS containing 2% BSA, and then reacted in a refrigerator overnight in a primary antibody solution. The next day, after washing 3 times every 5 minutes with a PBS-0.05% Tween 20 solution, it was reacted with a secondary antibody (Alexa Fluor 488- or 568-conjugated) (A11034; Invitrogen) at room temperature for 1-2 hours. Nuclei were mounted with a mounting solution containing DAPI for staining. A degree of fluorescence expression was observed using a confocal microscope (LSM 800, Carl Zeiss), and representative cells were selected and photographed.

Construction of Padi2 Knockout Cells Using the CRISPR-Cas9 Gene Editing System

Padi2 gene knockout by CRISPR-Cas9 gene editing was performed using the Padi2 Mouse Gene Knockout Kit (CRISPR) (Cat #KN512746; Origene) according to a manufacturer's protocol. After transfecting a Cas9-containing pCas-Guide plasmid including a guide RNA targeting a first exon of mouse Padi2 together with linear donor DNA including a LoxP-EF1A-tGFP-P2A-Puro-LoxP cassette to be used as a reporter for MC3T3-E1 cells, cell lines were established by receiving 1 cell/well of GFP-expressing cells in a 96-well plate using a single-cell fluorescence-activated cell sorting (FACS), and then growing them into cell clusters under puromycine (5 ug/mL) treatment. In the selected Padi2 knockout clones including #3-4 and #5-6, it was confirmed by Western blot that there was no expression at a protein level.

BrdU Cell Proliferation Assay

Cell proliferation was quantified by measuring absorbance at 450 nm using a Colorimetric BrdU Cell Proliferation ELISA Kit (ab126556; Abcam) according to the manufacturer's instructions. Data expressed BrdU incorporation as a fold change relative to a wild-type control. All experiments were repeated at least 3 times and expressed as mean±SD.

Statistical Analysis

To ensure data reliability, all experiments were performed as at least two or three independent experiments with three replicates and representative results are shown in figures. For statistical analyses, P values were calculated by unpaired two-tailed Student's t-test (when comparing only two groups) or one-way ANOVA or two-way ANOVA (when comparing more than two groups) in GraphPad Prism 9. All results are expressed as the mean±SD, and differences were considered significant at P<0.05. P values are as follows: *P<0.05; **P<0.01; ***P<0.001; ****P<0.0001.

<Experimental Example>

Promotion of an Osteoblast Senescence and Inhibition of an Osteoblast Differentiation Due to Excessive ROS.

After treatment with 50 μM or 100 μM H₂O₂ for 3 days to determine whether excessive ROS-induced oxidative stress promotes senescence in MC3T3-E1 cells, which is osteoblastic progenitor cells, as a result of staining with senescence associated-β-galactosidase (SA-β-gal) which is a marker of senescence, the number of SA-β-gal positive cells increased in a H₂O₂ concentration-dependent manner (A). In addition, it was shown that and a level of protein and an expression of cdkn1a (p21) mRNA which is a molecular marker of cellular senescence, were increased by H₂O₂ treatment (B and C). It was confirmed through ALP and Alizarin Res S staining for degrees of early osteoblast (increased alkaline phosphatase activity) and late differentiation (mineralization), respectively that that excessive ROS significantly inhibited a osteoblast differentiation function of MC3T3-E1 cells along with promotion of cellular senescence (D). These results suggest that excessive ROS induces function degradation of osteoblast along with osteoblast senescence.

Result of Verifying Whether Excessive ROS Reduces PADI2 in Mouse- and Human-Derived Mesenchymal Cells

When MC3T3-E1 cells were treated with 100 μM H₂O₂ for 1, 4, and 7 days in a differentiation medium, RNA-seq results were re-verified through RT-qPCR that the Padi2 mRNA level was significantly reduced (A). In addition, it was confirmed that an expression of PADI2 was significantly reduced by H₂O₂ treatment in human-derived mesenchymal stromal cells (hMSC) as well as mouse-derived MC3T3-E1 cells at a mRNA level as well as a protein level when 100 μM H₂O₂ treatment was performed (B). This suggests that decrease of PADI2 by excessive H₂O₂ is a common phenomenon occurring in human-derived cells. In addition, among five PADI isoforms (PADI 1, 2, 3, 4, and 6) through RT-qPCR, PADI2 was most predominantly expressed in MC3T3-E1 cells and hMSC, and an expression of the remaining isoforms was insignificant (C). Among the PADI isoforms, PADI2 is the most predominantly expressed isoform in mesenchymal cells, and is considered to play an important role in these cells. As a result of confirming an expression of PADI2 during osteoblast differentiation by Western blot, an expression of PADI2 was gradually increased as the differentiation of osteoblasts progressed, suggesting that PADI2 plays an important role in maturation of osteoblasts (D).

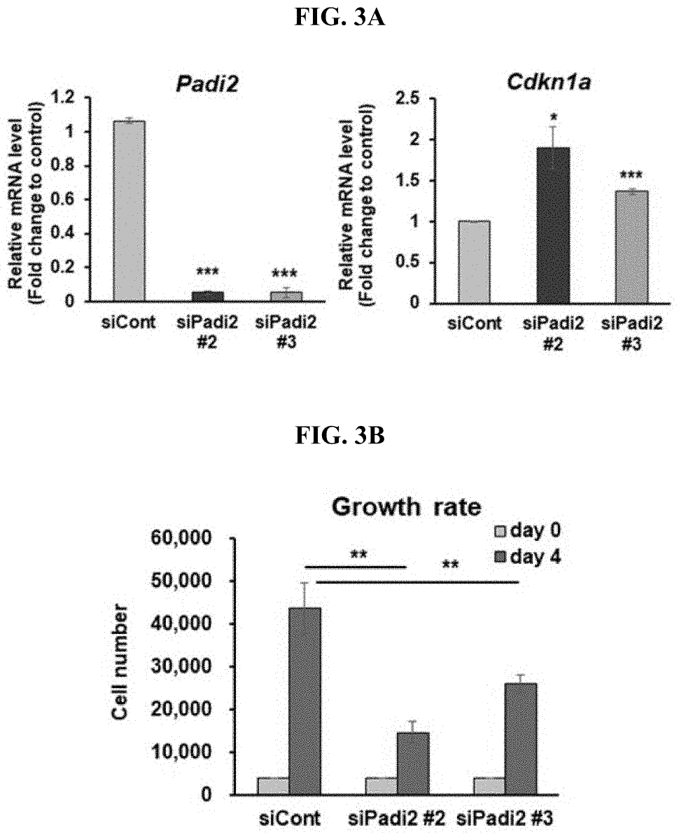

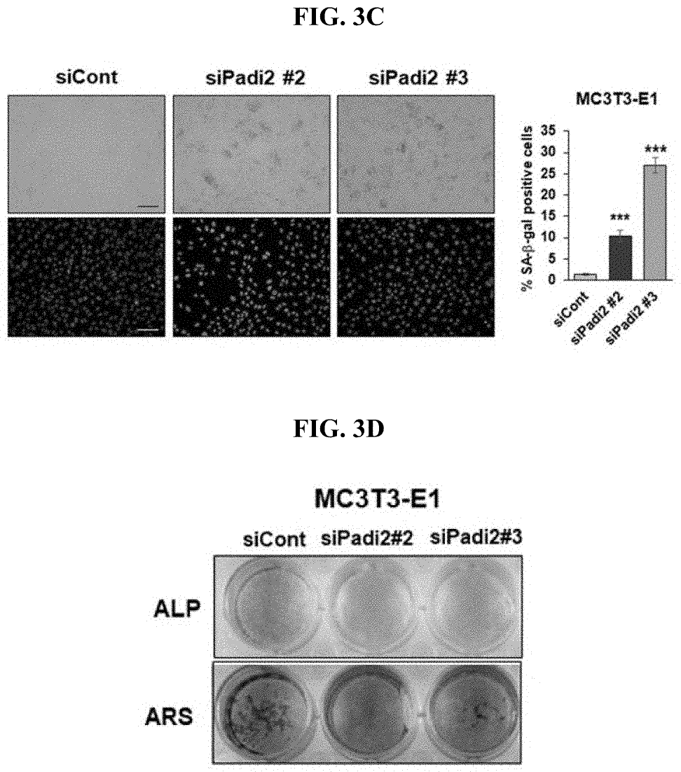

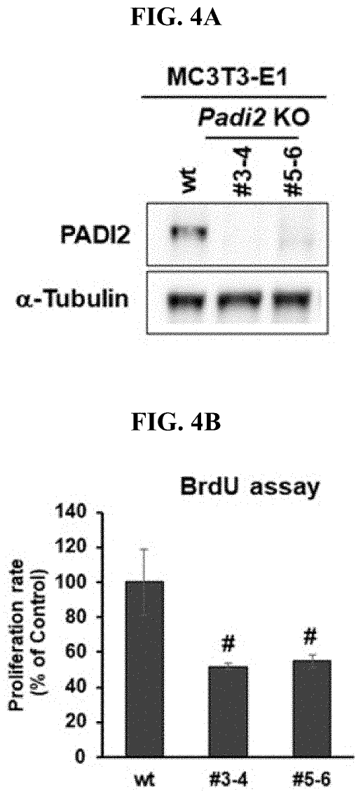

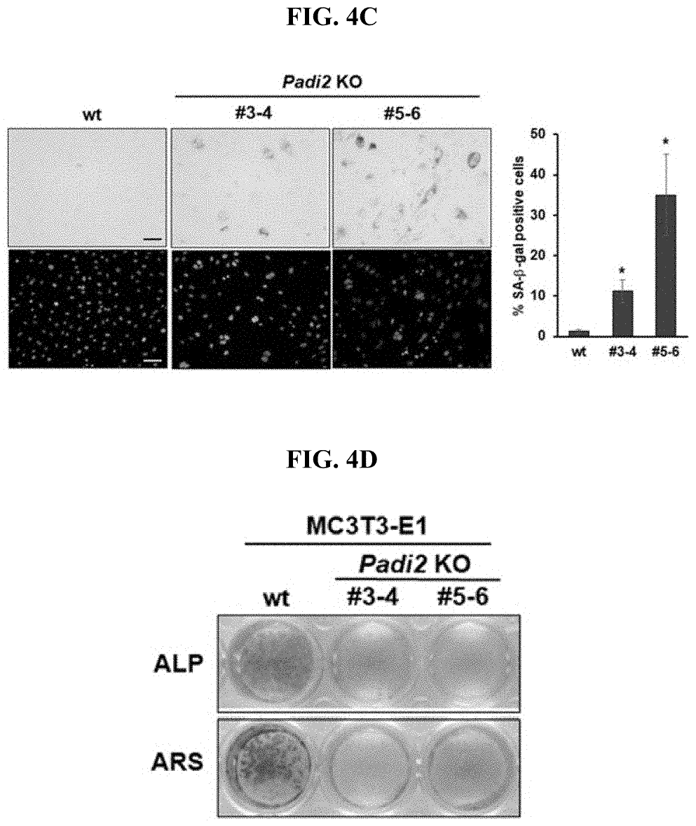

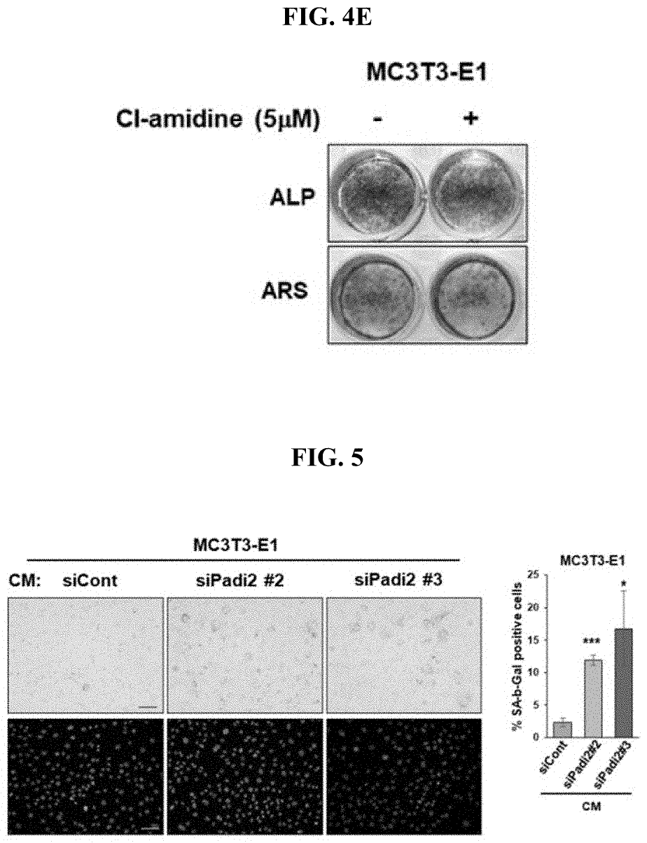

Promotion of Cellular Senescence and Reduction of Differentiation Function of Osteoblasts According to PADI2 Knockdown (FIGS. 3A-3D)

In order to determine whether decrease of PADI2 caused by excessive ROS directly induces promotion of cellular senescence, MC3T3-E1 cells were transfected with Padi2 siRNA to determine whether cellular senescence was promoted when Padi2 was knocked down. After transfection of two types of siRNA for Padi2, a knockdown effect of Padi2 was confirmed at a mRNA level, and a level of Cdkn1a mRNA, which is a cellular senescence molecular marker, significantly increased during Padi2 knockdown (A). Consistent with this, Padi2 knockdown reduced an MC3T3-E1 cell growth rate (B). The number of senescent cells was significantly increased in MC3T3-E1 cells subjected to Padi2 knockdown upon SA-b-gal staining which is a representative indicator of senescence (C). In addition, it was confirmed through ALP and ARS staining that a differentiation function of osteoblasts of Padi2-knockdown MC3T3-E1 cells was remarkably reduced (D). These experimental results show that decrease of PADI2 directly promotes cellular senescence and reduces a function of osteoblast.

Confirmation of Promotion of Cellular Senescence and Inhibition of Differentiation Function in PADI2 Knockout Cells Constructed by CRISPR-Cas9 Gene Editing Technology (FIG. 5)

Using CRISPR-Cas9 gene editing technology, it was reconfirmed whether cellular senescence was promoted as when Padi2 was knocked down in MC3T3-E1 cells. When compared with the control wild-type MC3T3-E1 cells, absence of PADI2 expression in the two Padi2 knockout cell lines was confirmed by Western blot (A). In addition, a growth rate of these knockout cell lines showed a significant decrease compared to a wild-type cell line (B). It was confirmed through SA-b-gal staining that the number of senescent cells was significantly increased in Padi2 knockout cell lines (C). Consistently, it was confirmed through ALP and ARS staining that a differentiation function of osteoblasts was significantly reduced when Padi2 was knocked out (D). Through these experimental results, it was reconfirmed once again that a decrease and a loss of PADI2 is an important factor in senescence promotion and cell functional degradation of osteoblast.

Promotion of Cellular Senescence by Factors Secreted from PADI2-Knockdown Cells (FIG. 5)

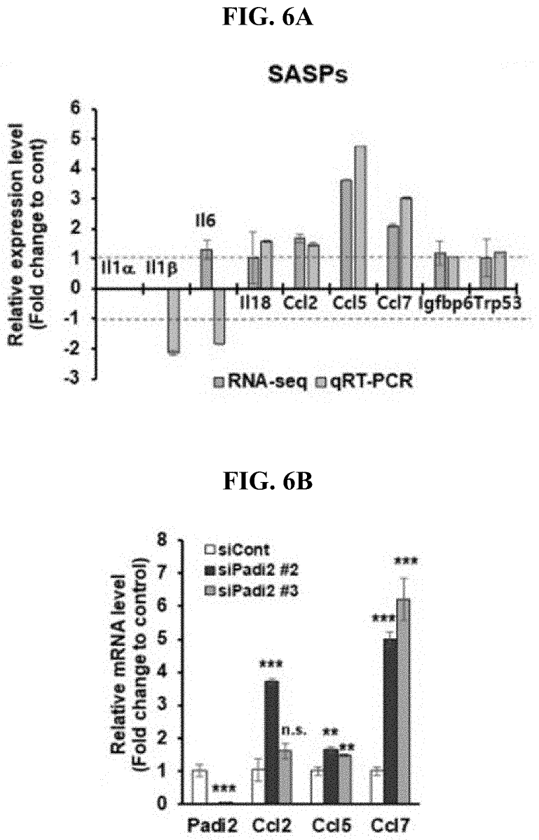

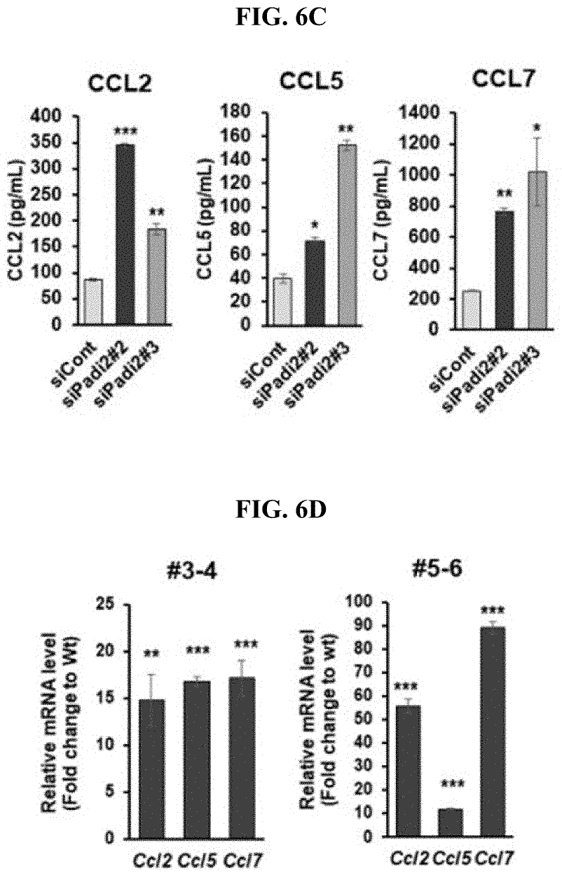

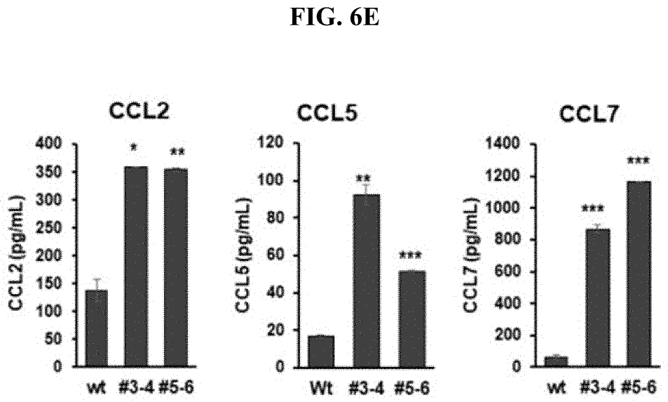

To determine whether a senescence-associated secretion phenotype (SASP) produced by Padi2 knockdown promotes senescence of surrounding cells in an autocrine and paracrine manner, when MC3T3-E1 cells were respectively cultured with these cultures after collecting Padi2 knockdown and siCont-transfected cell culture medium (conditional media, CM), the number of senescent cells was significantly increased in the group cultured in the Padi2-knockdown cell culture medium (FIGS. 6A-6E). This means that factors secreted from cells with reduced Padi2 accelerate cellular senescence.

Promotion of Expression and Secretion of SASPs (CCL2, CCL5, and CCL7) According to PADI2 Knockdown (FIGS. 6A-6E)

A senescence-associated secretion phenotype (SASP) is an inflammatory cytokine and chemokine secreted by senescent cells and is known to propagate and accelerate senescence to autologous and peripheral cells in an autocrine and paracrine manner. In the present invention, CCL2, CCL5, and CCL7 in SASP were significantly increased upon H₂O₂ treatment in RNA-seq and RT-qPCR verification (A), and when Padi2 was knocked down in MC3T3-E1 cells using siRNA, these CCL2, CCL5, CCL7 mRNA and secretion levels were significantly increased (B and C). Similarly, it was reconfirmed that CCL2, 5, 7 mRNA and secretion levels were significantly increased in the Padi2 knockout cell line compared to a wild-type cell line (D and E). Therefore, excessive ROS and a decrease of PADI2 in osteoblasts promotes the expression and secretion of CCL2, CCL5, and CCL7 among various SASP factors.

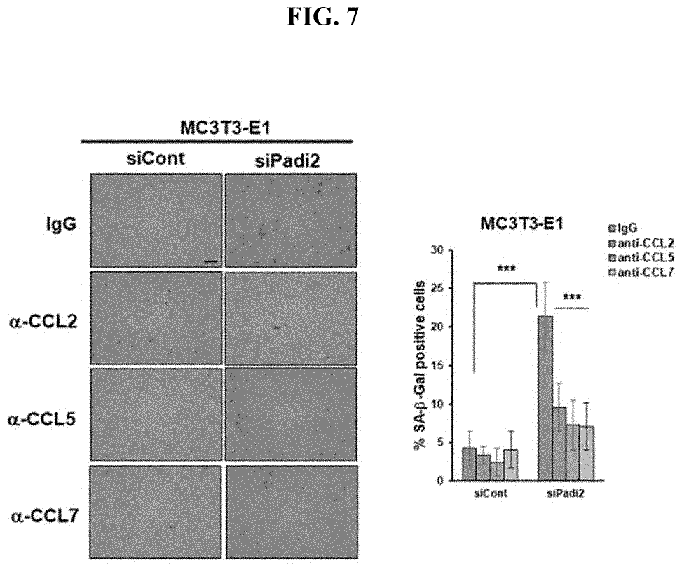

When CCL2, CCL5, and CCL7 were Inhibited Using an Antibody or siRNA, the Cellular Senescence and Decreased Function were Restored (FIGS. 7 & 9A-9D).

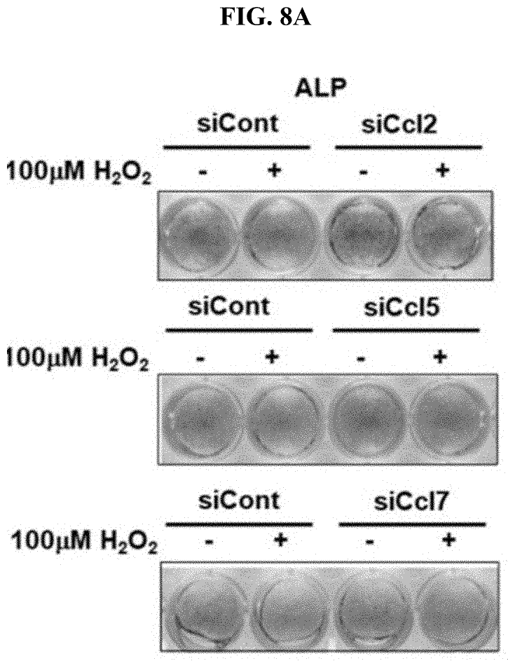

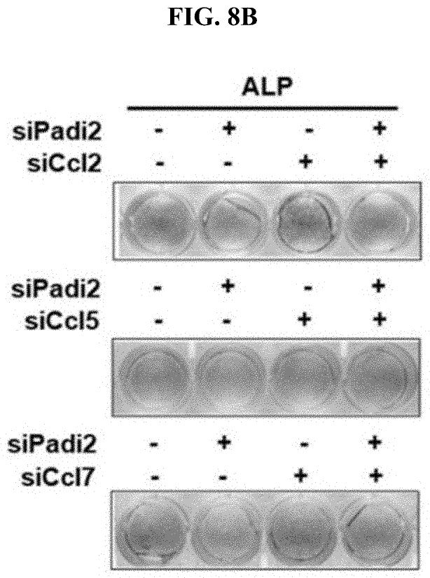

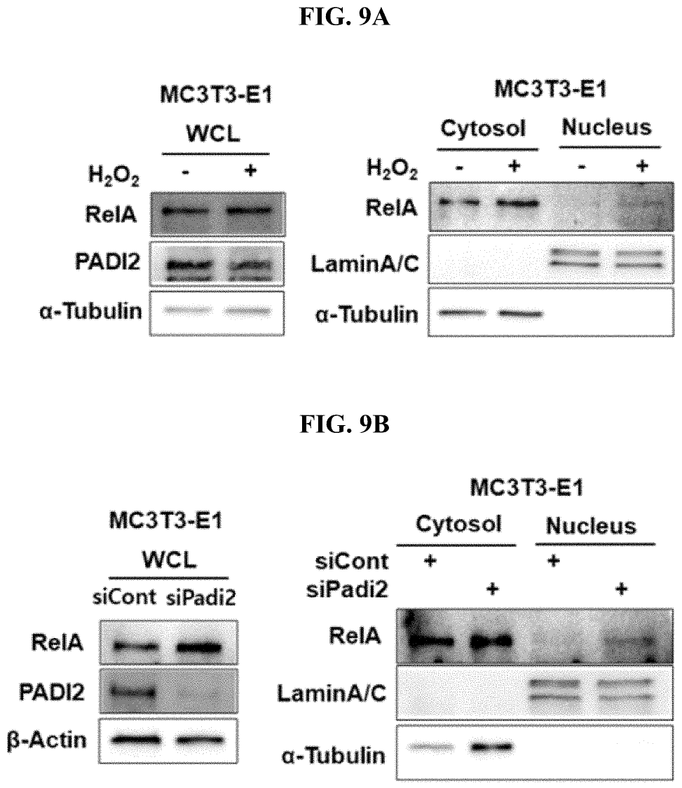

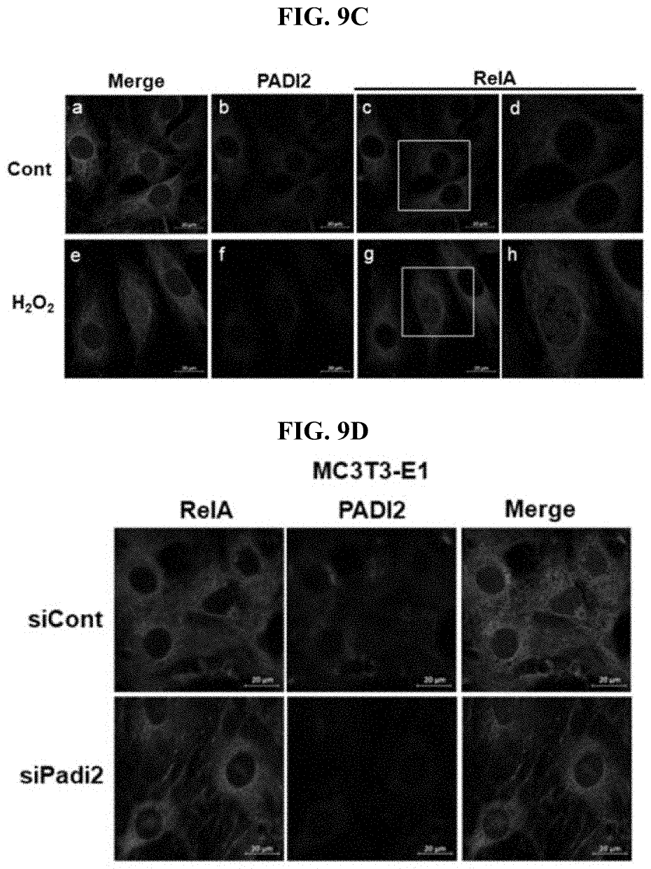

Whether cellular senescence promoted by Padi2 knockdown is due to an increase in CCL2, CCL5, or CCL7 was confirmed as follows. When an antibody and control IgG for each of these were treated for 4 to 5 days in MC3T3-E1 cells knocked out by Padi2, when compared to the control IgG treatment group, senescent cells increased by Padi2 knockdown were significantly reduced upon treatment of each of antibodies to CCL2, CCL5, and CCL7 (FIGS. 8A & 8B). In addition, it was confirmed through ALP staining that a differentiation function of osteoblasts deteriorated by H₂O₂ treatment or Padi2 knockdown was restored when these CCL2, CCL5, and CCL7 were knocked down with siRNA for each (FIGS. 9A & 9B). These results mean that among SASPs produced in osteoblasts by H₂O₂ or Padi2 knockdown, CCL2, CCL5, and CCL7 are main SASPs that cause osteoblast senescence and consequent functional deterioration.

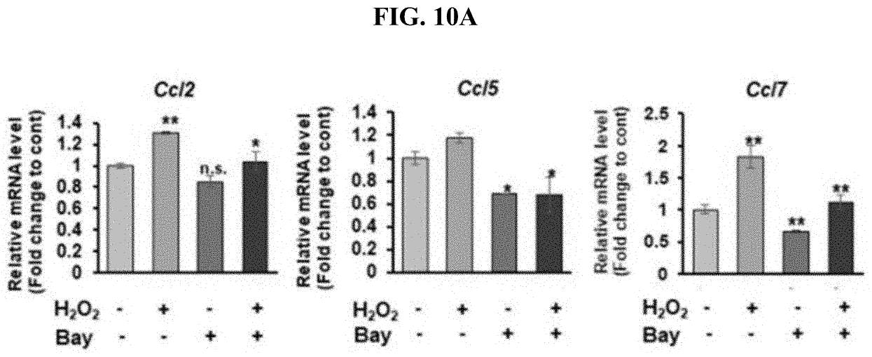

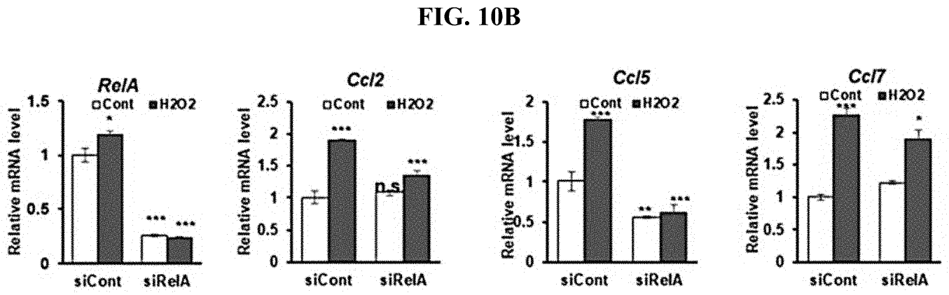

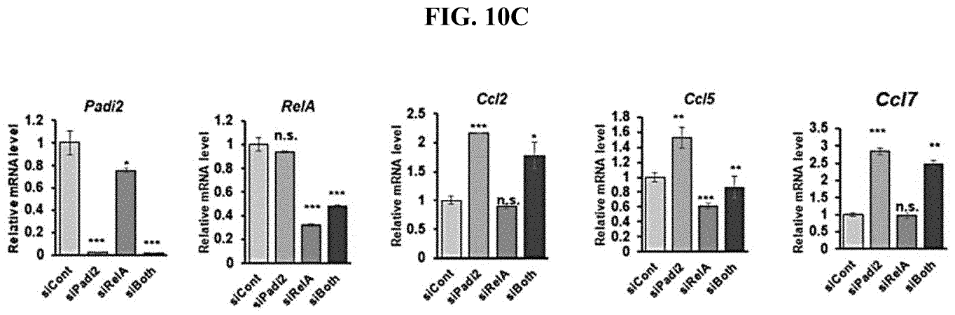

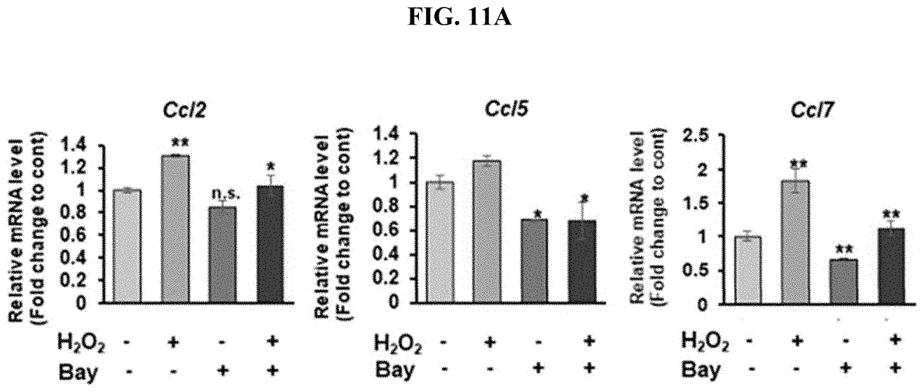

Activation of NFκB by Excessive ROS and PADI2 Knockdown Increases in Expressions of CCL2, CCL5, and CCL5 (FIGS. 9A-10D)

NFκB activation is known to be a major factor that increases an expression of SASP factor. Therefore, it was confirmed whether the NFκB signaling system mediated SASP expression increase caused by decrease of excessive ROS and PADI2. First, when MC3T3-E1 cells were treated with 100 μM H₂O₂ for 24 h, an NFκB RelA transcription factor was activated, and thus Western blot and Immunofluorescence confirmed whether the NFκB RelA transcription factor is activated and transfers from cytoplasm to nucleus, and RelA was activated by H₂O₂ treatment and migration to the nucleus was observed (FIGS. 10A and 10C). Similarly, RelA transferred from the cytoplasm to the nucleus by Padi2 knockdown (FIGS. 10B and 10D). Through this, it was confirmed that reduction of excessive ROS and Padi2 in osteoblasts activates NFκB.

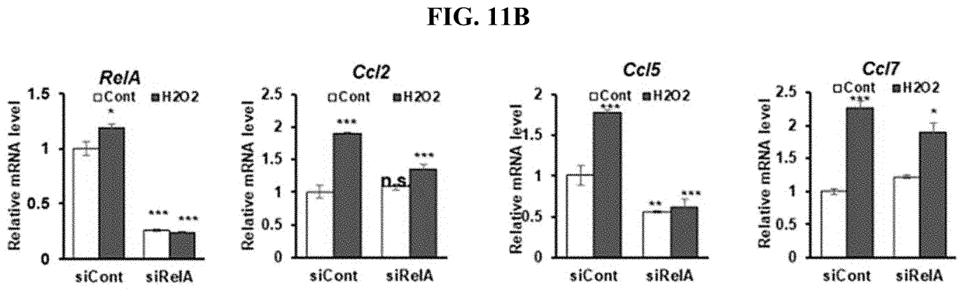

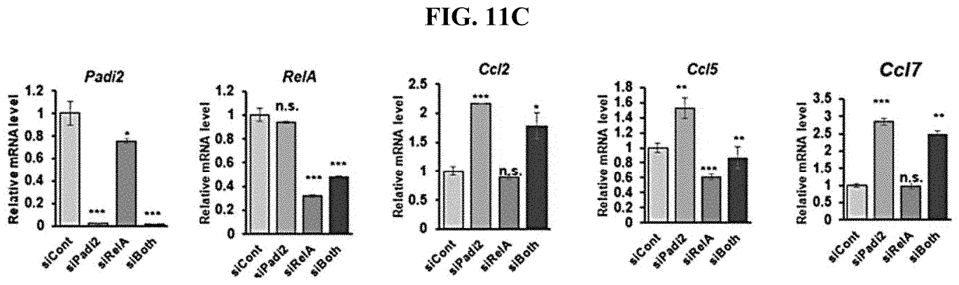

Next, in order to determine whether NFκB mediates the increase in expressions of CCL2, CCL5, and CCL7 caused by H₂O₂ or Padi2 knockdown, as a result of checking the CCL2, CCL5, and CCL7 mRNA levels after using H₂O₂ and Bay11-7082, which is an NFκB inhibitor, in combination or alone, expressions of CCL2, CCL5, and CCL7 increased by H₂O₂ was significantly reduced when Bay11-7082 was combined with treatment. (FIG. 11A). Similarly, CCL2, CCL5, and CCL7 mRNA levels increased by H₂O₂ were decreased upon RelA knockdown (FIG. 11B). CCL2, CCL5, and CCL7 increased by Padi2 knockdown were also significantly decreased when RelA was knocked down together (FIG. 11C). These results suggest that reduction of excessive ROS and Padi2 activates NFκB and increases expressions of CCL2, CCL5, and CCL7.

Restoration of Cellular Senescence and Function Degradation of Osteoblast Due to Excessive ROS and PADI2 Knockdown Upon Inhibition of NFκB Signaling

Since the increase in expressions of CCL2, CCL5, and CCL7 caused by excessive ROS or Padi2 knockdown occurs through NFκB activation, it was checked whether cellular senescence promoted by decrease of PADI2 caused by excessive ROS was alleviated when NFκB signaling was inhibited. When 1 μM Bay11-7082, which is an NFκB inhibitor, was co-treated with 100 μM H₂O₂, the number of senescent cells increased by H₂O₂ alone was reduced and an expression of Cdkn1a, which is a senescence molecular marker was also reduced (A and B). In addition, when senescent cells increased by Padi2 knockdown were treated with Bay11-7082, the number of senescent cells and Cdkn1a expression were decreased (C and D). Similarly, it was confirmed that the number of senescent cells and cdkn1a expression increased by Padi2 knockdown were decreased even when Padi2 and RelA were knocked down simultaneously (E and F). Consistently, it was confirmed through ALP and ARS staining that inhibition of NFκB signaling using NFκB inhibitors or RelA siRNA restored function degradation of osteoblast caused by excessive ROS and Padi2 knockdown (G, H, I, and J). This suggests that the NFκB inhibition method can be utilized to alleviate senescence caused by excessive oxidative stress and decrease of PADI2 or to treat age-related diseases related thereto.

Comparison of PADI2 mRNA Expression in Mesenchymal Stem Cells (hMSCs) Derived from Young and Elderly Humans

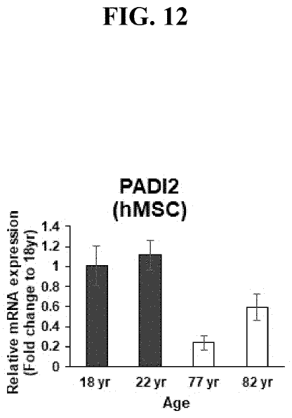

Referring to FIG. 12, when PADI2 mRNA levels in 18- and 22-year-old hMSCs and 77- and 82-year-old hMSCs were compared by RT-qPCR, PADI2 mRNA levels in the elderly group tended to be lower than in the young group. Although statistical significance cannot be obtained due to a small number of samples, it strongly suggests that PADI2 expression decrease may be associated with the senescence process.

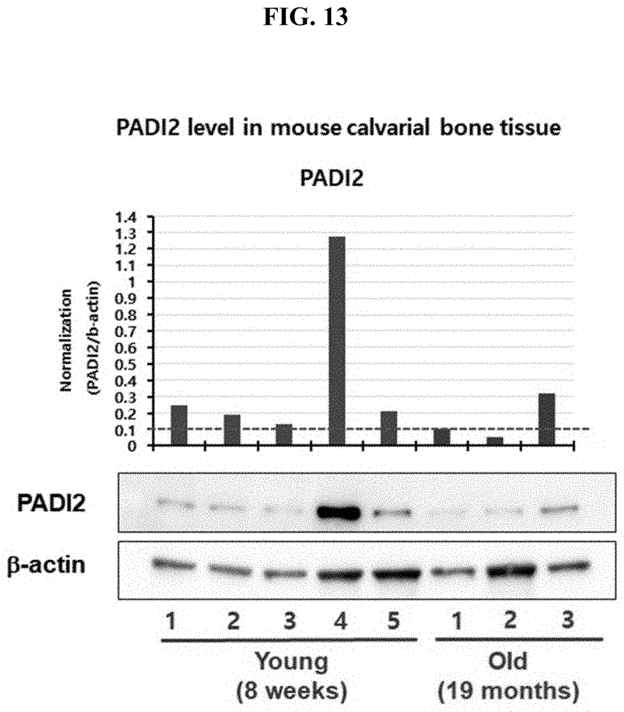

Comparison of PADI2 Protein Levels in Young and Old Mouse Cranial Tissues (FIG. 13)

The expression level of PADI2 in mouse cranial bone of 8 weeks old (n=5) and 19 months old (n=3) was confirmed by Western blot. There was a difference in the expression level of each individual and the number of samples was low, but the statistical significance was low, however, when corrected with b-actin, the expression of PADI2 was lower in the old mouse group than in the young mouse group. This result also strongly suggests that PADI2 expression decrease may be associated with the senescence process.

The present invention proposes a mechanism by which excessive ROS promotes osteoblastic senescence in terms of cellular senescence as a major factor in senescence and as a model of senescence at a cellular level, and a technology capable of controlling the mechanism. The excessive ROS in osteoblasts caused a decrease in the posttranslational modification enzyme, peptidyl arginine deiminase 2 (PADI2) which converts peptidyl arginine to citrulline and promoted senescence and functional deterioration of osteoblasts. That is, osteoblast senescence was promoted, and a differentiation function of osteoblasts was significantly reduced upon PADI2 knockdown by H₂O₂ administration or RNAi technique. In addition, H₂O₂ or PADI2 knockdown increased the expression and secretion of CCL2, CCL5, and CCL7, known as SASP factors secreted by senescent cells, and when they were blocked with antibodies against them, osteoblast senescence promoted by H₂O₂ administration or PADI2 knockdown was alleviated. In addition, H₂O₂ administration and PADI2 knockdown activated a NFκB signaling pathway to increase the expression and secretion of SASP, thereby promoting osteoblast senescence. When the NFκB signaling system was inhibited using an inhibitor or siRNA, senescence promotion and cell functional deterioration of osteoblast caused by H₂O₂ administration and PADI2 knockdown were effectively restored. According to the present invention, a role of PADI2 in the process of promoting osteoblast senescence due to excessive ROS and novel biomarkers and treatment methods for senescence and age-related bone diseases may be provided.

In the present invention, it was discovered that decrease of PADI2 caused by oxidative stress promotes cellular senescence, resulting in functional deterioration of osteoblasts. In addition, it was revealed that cellular senescence and function degradation of osteoblast due to excessive ROS and a decrease in PADI2 occurred through the increases in the expression and secretion of SASP factors, CCL2, CCL5, and CCL7 by activation of NFκB. Thus, it is suggested that PADI2 plays a role as a key regulator of osteoblast senescence due to excessive ROS. In addition, the present invention proposes targets for the development of therapeutic agents for anti-senescence and age-related diseases by elucidating a novel regulatory mechanism of cellular senescence.

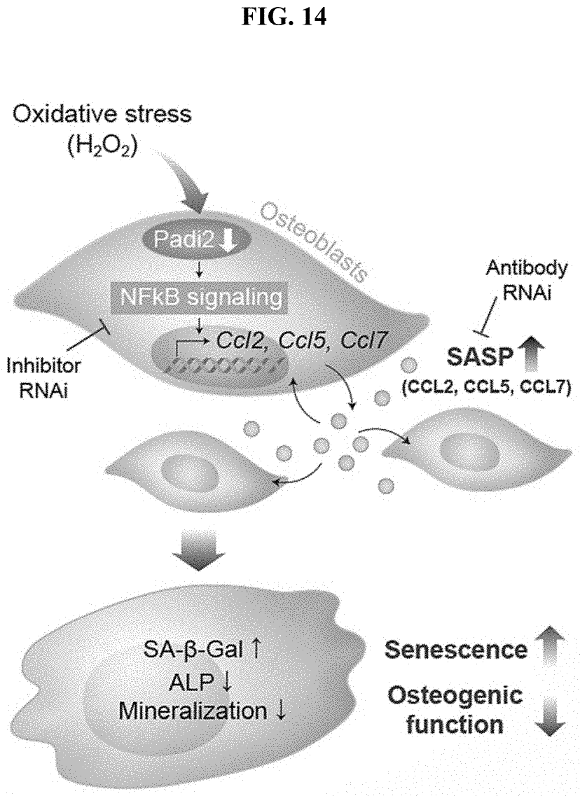

FIG. 14 is a schematic diagram of describing the mechanism of cellular senescence and functional deterioration of osteoblasts by the decrease of PADI2, and preventive and therapeutic targets for senescence and aging-associated diseases.

As described above, the present invention has proven through various experimental methods that when PADI2 is reduced, it promotes cellular senescence and promotes function degradation of osteoblast, which is a new discovery that has not been previously reported, and it was revealed that the decrease in PADI2 promotes cellular senescence by activating nuclear factor kappa-light-chain-enhancer of activated B cells (NFκB) signaling to increase expression and secretion of SASP factors of CCL2, CCL5, and CCL7. Therefore, it is determined that decrease of PADI2, increases in CCL2, CCL5, and CCL7, and the activation of NFκB signal pathway can be utilized as senescence markers based on the present invention.

In addition, in the case of aging-related diseases caused by decrease of PADI2, it is expected that a method of inhibiting CCL2, CCL5, and CCL7 or inhibiting NFκB signaling can be utilized for disease treatment.

In the present specification and drawings, preferred embodiments of the present invention have been disclosed, and although specific terms are used, these are only used in a general sense to easily explain the technical content of the present invention and help the understanding of the present invention. It is not intended to limit the scope. It will be apparent to those skilled in the art that other modifications based on the technical concept of the present invention can be performed in addition to the embodiments disclosed herein.

### Effects of the Invention

The present invention can utilize the decrease of PADI2, the increases in CCL2, CCL5 and CCL7, and NFκB activation as biomarkers for senescence and aging-related diseases, and as surrogate markers capable of evaluating efficacies of anti-senescence agents and therapeutic agents for age-related diseases.

The present invention can utilize an antibody or siRNA capable of inhibiting CCL2, CCL5, and CCL7 for treatment of age-related diseases caused by decrease of PADI2.

The present invention can utilize inhibitors of NFκB signaling pathway for treatment of senescence and aging-related diseases caused by decrease of PADI2.

Specifically, as a composition for preventing or alleviating cellular senescence according to one embodiment of the present invention, the composition including the PADI2 can prevent or alleviate cellular senescence by inhibiting secretion of inflammatory cytokines, growth factors, chemokines, or proteases in cells or inhibiting an activity of NFκB.

According to another embodiment of the present invention, it is possible to improve a differentiation function of osteoblasts by providing PADI2 to osteoblasts.

According to another embodiment of the present invention, it is possible to alleviate or treat senescence or age-associated diseases caused by decrease of PADI2 through antibodies of CCL2, CCL5 and CCL7.

According to another embodiment of the present invention, it is possible to alleviate or treat senescence or age-associated diseases caused by decrease of PADI2 through an NFκB inhibitor.

According to another embodiment of the present invention, it is possible to alleviate or treat senescence or age-associated diseases caused by decrease of PADI2 by administering antibodies of CCL2, CCL5 and CCL7 to a subject.

According to another embodiment of the present invention, it is possible to alleviate or treat senescence or age-associated diseases caused by decrease of PADI2 by administering an NFκB inhibitor to a subject.

According to another embodiment of the present invention, a diagnostic kit capable of diagnosing a decrease of PADI2 including detectable labeled antibodies of CCL2, CCL5 and CCL7 may be provided.

According to another embodiment of the present invention, a diagnostic kit capable of diagnosing a decrease of PADI2 including a detectable labeled NFκB inhibitor may be provided.

The effects of the present invention are not limited to those mentioned above, and other effects not mentioned may be clearly understood by those skilled in the art from the following description.

This research was supported by Basic Science Research Program through the National Research Foundation of Korea (NRF) funded by the Ministry of Education (Hyun Mo Ryoo (2020R1A4A1019423), Hyun Jung Kim (2021R1A2C1007715), and Woo Jin Kim (2019R1C1C1003669)).

What is claimed is:

1. A method for preventing or alleviating aging of a subject comprising administering a composition comprising a peptidylarginine deiminase 2 (PADI2) to the subject.

2. The method of claim 1, wherein the composition inhibits production or secretion of inflammatory cytokines, growth factors, chemokines, or proteases in cells.

3. The method of claim 1, wherein the composition inhibits activation of NFκB signaling pathway.

4. A method for improving function of osteoblasts in a subject comprising administering a composition comprising a peptidylarginine deiminase 2 (PADI2) to the subject.

5. A method for alleviating or treating a decrease of peptidylarginine deiminase 2 (PADI2) in a subject comprising administering to the subject, a composition comprising:
(a) a composition comprising an anti-CCL2 antibody, an anti-CCL5 antibody, an anti-CCL7 antibody, or a combination thereof; and or
(b) an NFκB inhibitor.

6. A diagnostic kit for a decrease of peptidylarginine deiminase 2 (PADI2) in a biological sample, including
(a) an anti-CCL2 antibody, an anti-CCL5 antibody, an anti-CCL7 antibody, or a combination thereof; and or
(b) an NFκB inhibitor.

7. A method for detecting a decrease of peptidylarginine deiminase 2 (PADI2) in a biological sample, comprising
(a) contacting the biological sample with an anti-CCL2 antibody, an anti-CCL5 antibody, an anti-CCL7 antibody, or a combination thereof, wherein the anti-CCL2 antibody, the anti-CCL5 antibody, and the anti-CCL7 antibody comprise a detectable marker;
(b) contacting the biological sample with an NFκB inhibitor, wherein the NFκB inhibitor comprises a detectable marker; and/or
(c) measuring an activation of NFκB signaling pathway or transactivation activity of NFκB of the biological sample.
